(19) United States
(12) Patent Application Publication
(10) Pub. No.: US 2006/0025680 A1
(43) Pub. Date: Feb. 2, 2006

(54) Method and apparatus for contrast enhanced medical imaging

(76) Inventors: Fanny Jeune-Iomme, Versailles (FR); Serge Louis Muller, Guyancourt (FR); Razvan Gabriel Iordache, Paris (FR)

Correspondence Address: CANTOR COLBURN, LLP, 55 GRIFFIN ROAD SOUTH, BLOOMFIELD, CT, 06002, US

(21) Appl. No.: 11/114,379

(22) Filed: Apr. 26, 2005

(30) Foreign Application Priority Data
Jul. 29, 2004 (FR) 04 08380

(51) Int. Cl. A61B 5/05

(52) U.S. Cl. 600/425

(57) ABSTRACT

A method and apparatus for breast vascularization from projective images resulting from the attenuations of an X-ray beam across the breast in the presence of an injected contrast-medium product. A series of projective images is acquired for different orientations of the beam, in relation to the breast. The projective images are treated to provide a reconstruction of a three-dimensional model of the breast observed.

### CROSS-REFERENCE TO RELATED APPLICATIONS

This application claims the benefit of a priority under 35 USC 119(a)-(d) to French Patent Application No. 04 04380 filed Jul. 29, 2004, the entire contents of which are hereby incorporated by reference.

### BACKGROUND OF THE INVENTION

An embodiment of the invention concerns medical imaging, particularly a method and apparatus for mammography using radiation, such as X-rays.

An embodiment of the method and apparatus is directed to medical imaging of an object aided by contrast enhancement. An embodiment of the invention is directed to an increased visibility of an intended image of an animate or inanimate object, such as a human body organ or feature. The increased visibility may for example, reveal in the object such internal features as discontinuities, voids, cracks, lesions and tumors. An embodiment of the invention concerns mammography, in particular when it is used to make vascularization, i.e., increased visibility, of a breast apparent by injection of contrast-enhancement such as an iodized product or medium. The injection of contrast-medium in X-ray imaging, particularly in a mammography, is a well-known technique. In particular tumors present a vascularization which is much more dense than healthy tissue, and the contrast-medium fills more quickly and with a stronger concentration in these areas. Moreover, X-ray imaging and injection of the contrast-medium present a weak ratio in contrast to the noise of the signal of the iodine. X-ray imaging, in particular when associated with the injection of contrast-medium, therefore allows for an identification of the tumor and its location.

However X-ray imaging retains a certain limitation in so far as the images obtained are projective images. In other words, these images represent the reduction of the beam of radiation, each dot of the image corresponding to the crossing of the whole breast, in the thickness of the tumor and the other tissue. US 2002/0003861 A1 describes a system that enables the visualization of the enhancement in breast tissues in 2D.

The known X-ray imaging using a contrast-medium has disadvantages due to a number of reasons, such as the difficulty to provide for a high acquisition rate by the need to change the film and the difficulty to process the acquired films.

It has also been suggested to use a contrast medium or agents in magnetic resonance imaging that provides a 3D model of the breast.

### BRIEF DESCRIPTION OF THE INVENTION

An embodiment of the invention is an apparatus to improve the efficiency of the means of observation existing in the subject matter of the mammography by X-rays and injection of the contrast-medium (CMM-Contrast Medium Mammography). In an embodiment of the invention, a breast is observed by sampling of projective images resulting from reductions of a beam of X-rays at the crossing of the breast in the presence of the injected contrast-medium. A series of projective images is taken from different orientations of beams in comparison with the breast, and the projective images are processed by a reconstruction of a three-dimensional (3D) model of the observed breast.

An embodiment of the invention is directed to a method of observation of a reduction of the X-ray beams at the crossing of a breast in the presence of an injected contrast-medium. A series of projective images of reduction of the beam from different orientations in comparison with the breast, and processing the projective images comprising a reconstruction of a visual three dimensional (3D) model of the breast.

An embodiment of the invention will be better understood after reading the detailed description that follows, made with reference to the related figures of which:

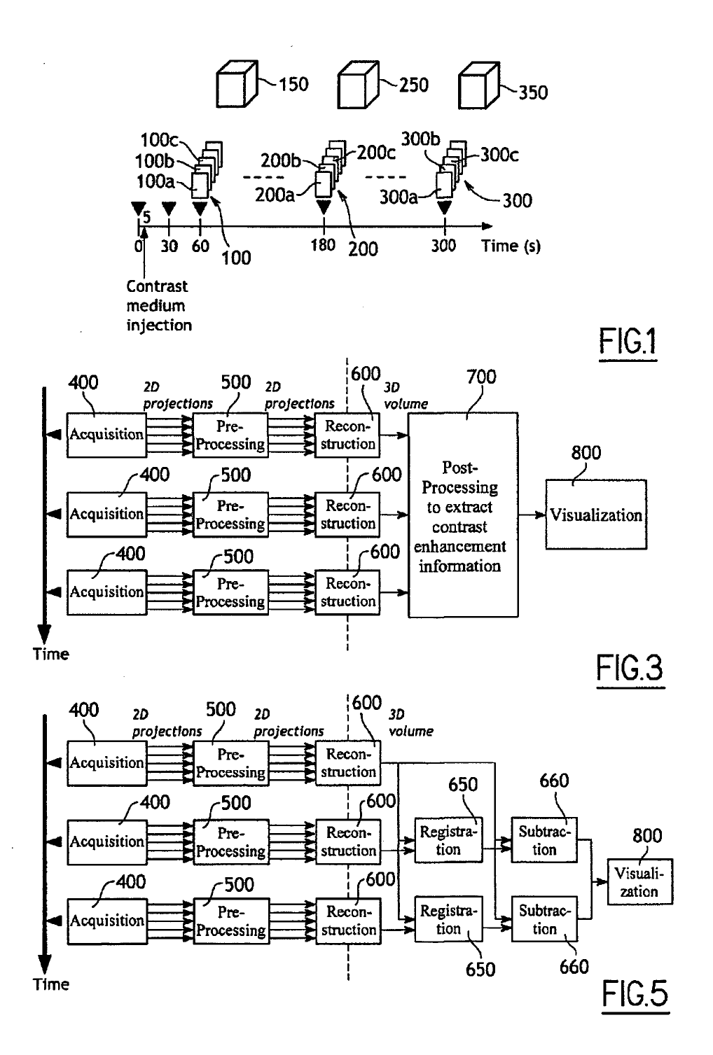

FIG. 1 is a diagram giving an example of temporal distribution of the different stages of a method in accordance with an embodiment of the invention;

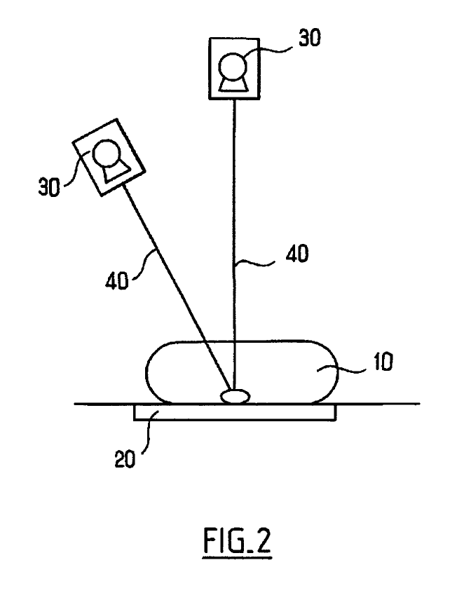

FIG. 2 is a diagram representative of a method for image acquisition in accordance with an embodiment of the invention;

FIG. 3 is a representative flow chart for making use of an embodiment of the invention;

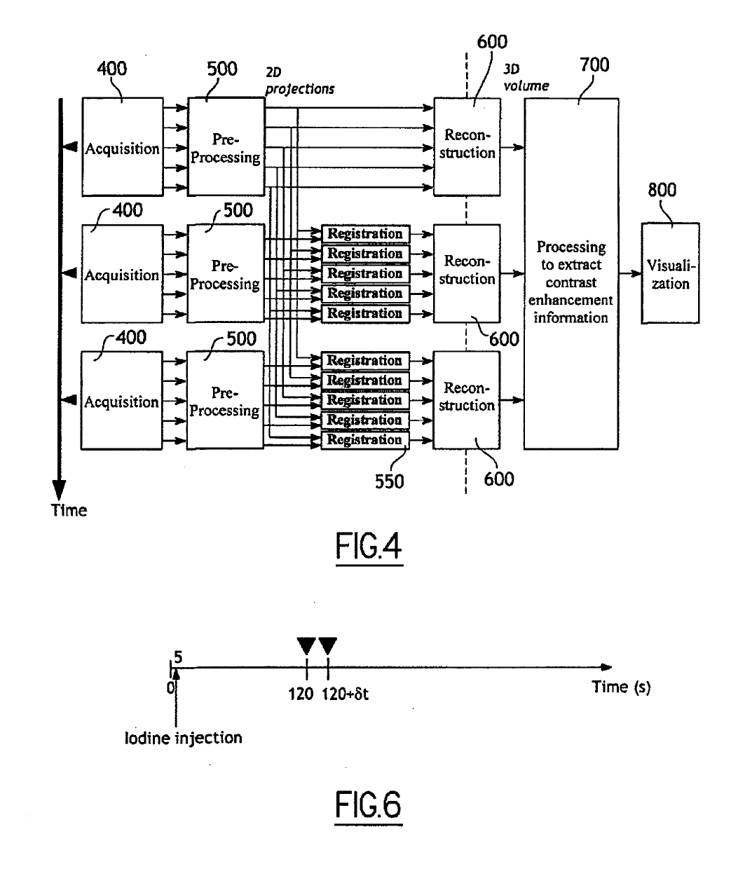

FIG. 4 is a flow chart corresponding to a preliminary retiming variant embodiment of the invention;

FIG. 5 is a flow chart corresponding to a later variant of retiming for image reconstruction according to an embodiment of the invention; and

FIG. 6 is a timing diagram for a dual energy embodiment of the invention.

### DETAILED DESCRIPTION OF THE INVENTION

An embodiment of the invention applies a Full Field Digital Mammography System (FFDM). FFDM permits obtaining a large series of images during the insertion of the contrast-medium in the breast, in such a way that this series of images illustrates the contrast-medium as it progresses in time.

As shown in FIG. 1 the acquisition stage of each series of 100, 200, 300 images of which each one serves as an elaboration of a geometric model corresponding to 150, 250, 350, is carried out in the following way. The breast 10 is placed, as shown in FIG. 2, below a means for sensing 20, that may be fixed, and underneath a means for radiation, such as a source 30, rising and/or rotating below the sensor 20, for example on a pivotal arm 40. This acquisition of a series of images of projections is optimized with regard to selected parameters of radiation appropriate to a desired protocol. Typically the beam of radiation is of a strong intensity, that is to say having been subject to a strong acceleration tension/voltage, typically above 45 kVp.

The energy or frequency of radiation is positioned in an optimal way by use of a wide (thick) filter, that is to say typically a thickness of 0.5 mm, based on copper or zinc. A spectrum is obtained which is as close as possible to the optimal spectrum of a source of monochromatic X-rays whose energy is situated just below the start of the parameter of reduction (k-edge) of an iodized contrast agent such as that which can be used in an embodiment of the invention. This optimization of the beam establishes the average energy of the spectrum to make apparent in the best way, the differences of contrast between the enhancement medium, such as iodized product, and tissue. As a result of this optimization, a global dose of radiation is sufficiently limited to allow a multitude of exposures to be taken of the person to be imaged.

A pre-recorded table indicates optimal parameters of radiation working on different parameters of the image acquisition. These acquisition parameters on which the optimal parameters depend, are for example, the thickness of the observed breast and the apparent composition as well as the compression value that is applied to the breast.

The first geometric model 150 represents the breast when it is not filled with any contrast-medium product. This first 3D model will be referred to here as the mask. Subsequently the same term will be used to denote the projection images of the series having attended to its reconstruction.

The acquisition of a series of images 100a, 100b, 100c, . . . 200a, 200b, 200c, . . . 300a, 300b, 300c . . . corresponds therefore to a progressive swing of the arm 40 and of the source 30 around the breast 10 that is placed in a fixed position on the sensor 20.

The reconstruction used here is carried out by means of geometric processing of the projections on the sensor 20, the 3D model being deduced under an approximation that would have given a series of identical projections to the one removed. Before the reconstruction of each geometric model 150, 250, 350, a retiming of each of the projection images 100a, 100b, 100c, is carried out in order to compensate for an eventual non-voluntary movement of the breast 10 in relation to the sensor 20 that may cause artifacts in the images acquired or the contrast-medium uptake quantification. In effect, in the course of the insertion of the contrast-medium product, the patient may have moved slightly and/or the means of acquisition can present an undesirable geometric gap.

In a variant embodiment, this spatial retiming can take place after reconstruction of each of the 3D models, that is to say, on each of the 3D models 150, 250, 350.

The recorded contrast and its reduction are then exploited for as much information as possible in order to reveal an eventual pathology. Therefore, at this stage, a retiming of each 3D volume of the sequence in relation to the “mask” volume, is carried out as a way of compensating an eventual movement of the patient during the acquisition.

In the variant embodiment, the retiming can be applied to the mask volume itself, in relation to each of the consecutive volumes.

A reconstruction algorithm takes account of the intensity recorded, particularly after application of a logarithmic function to the intensity values such that they appear on the sensor 20. In effect, the logarithm of the recorded reduction is noticeably proportionate to the concentrations of the contrast-medium product met by the beam in the course of its perfusion. These logarithmic values can be associated with the values of medium concentration present in the breast. These values, as a result of three-dimensional reconstruction can be distributed in-the volume of the three dimensional model. Positioned in this way for example under the form of shade more or less raised in function of the corresponding values, these values can be consulted in an assessment by the general practitioner. In a variant embodiment, the 3D model can be constructed in a non-linear way, by processing intending to provide an indication specifically illustrative of contrast-enhancement visualization, that is to say particularly aesthetically revealing of the image.

A sequence of models 150, 250, 350 and 3D (volumes) each representing a progressive contrast is obtained. Each volume of the sequence undergoes a processing to extract the contrast enhancement signal information. The processing may comprise one or several treatment steps. For example, the subtraction or taking away, at each geometric point, of the contrast enhancement signal at this same point for the mask volume (stage 700 in FIG. 4). Thus, the subtraction eliminates in each volume the signals which are the signals present from the start and which are present across the different tissue. After this subtraction, there only remain the contrast enhancement signals due exclusively to the presence of the contrast-medium product. The volume obtained after subtraction therefore illustrates exclusively the vascularization of the breast.

The physical value of the reduction factor of the contrast medium that was used is known as well as the spectrum of radiation of the beam from source 30. Further, for example, from this known information, it is possible to estimate the attenuation factor, the effective value of the concentration in attenuation product for each localization in the represented volume. In each subsequent 3D model during the immersion, the value of concentration in iodized attenuation product is illustrated (for example in mg/cm³). The set of these 3D models, associated to contrast values and/or values relative to the concentration in iodized products, is therefore used as a fully representative immersion sequence. A treatment of this contrast progression can be implemented to provide kinetic information in each localization. This treatment can provide a 3D model illustrating the distribution of the kinetic values of the contrast images in the breast volume. Thus this 3D model for example, illustrates the scale of the maximal slope of contrast progression in each point, through more or less dark zones. Another kinetic parameter that can be illustrated is the value of the contrast temporal entirety for each considered breast point.

Still further, for example, a spatial filtering of each 3D model (before or after subtraction) can be applied in order to increase the noise-induced contrast ratio, especially in the areas that tend to correspond to lesions. These zones will be typically larger than the height of the detector picture element.

In addition, for example, the processing of the 3D volumes in order to extract the contrast enhancement information can be provided by computation of parametric images (e.g., maximum gradient of increase, positive enhancement integral known from CT/MRI imaging) to characterize the contrast-variation.

The treatment can be provided by means for information technology from a computer program able to carry out these various treatments. The computer program is can be provided with supervised learning methods, apt to take into account reference data corresponding to certain pathologies introduced in a memory of the apparatus. The apparatus compares the reference data with the data encountered in the 3D model, so as to identify any potential similarities or connections between the acquired values and the reference values, and to issue a potential diagnosis, such as, for example the identification of certain pathologies. This means of automatic diagnostics for example, are suitable for the acquisition of certain reference data in the form of previously entered data files. They can also comprise means for acquiring the reference data as results of different diagnoses carried out during the apparatus usage, results issued by the apparatus or by a practitioner.

FIG. 3 is a general flow chart corresponding to the previously described different treatment stages. The acquisition stage is represented as 400; the pre-treatment stage is represented as 500; and the reconstruction stage is represented as 600. Each line in the flow chart represents the reconstruction of a different 3D model in the sequence of such models. Stage 700 corresponds to the post-treatment issuing the kinetic information pertinent to the contrast progression. Stage 800 is the stage relative to the visualization of the 3D models and of the associated results.

FIGS. 4 and 5, indicate the positioning of the retiming stage, respectively when the retiming is carried out before (FIG. 4) and after (FIG. 5) the reconstruction stage 600. In FIG. 5, stage 650 is the stage relative to the subtraction of contrasts between the reconstructed model and the mask. The pre-treatment stage 500 can be likewise applied to all operation types and embodiments able to improve the noise-induced contrast ratio upon each view projected before the reconstruction, for example by subtraction, spatial filtration or temporal retiming. The reconstruction of the 3D model can also be applied to projection images in all embodiments that are already the object of a subtraction process and of a logarithmic treatment. The sequence of 3D models representative of the contrast image can hence be illustrated in comparison with a display screen, with the model forming the mask.

In a variant embodiment, the breast can be exposed to a double radiation exposure according to two different radiation energies, so as to better reveal the contrast image. To attain a same beam orientation, the breast is exposed to two different radiation energies (frequencies) at two very close intervals. It is well known that the contrast-medium product behaves differently in terms of attenuation according to the radiation energy to which it is subjected. Thus, by subtracting the two obtained projection images, the specific influence of the contrast-medium product can be deduced. The interval separating the two associated acquisitions should be as short as possible, in order to avoid at the very most, all inaccuracies due to the patient being transferred or motion induced artifacts. The subtraction between these two acquisitions can be replaced by a weighted subtraction, defined so as to optimize the visibility of the iodized product, or even by a more elaborated combination of each obtained projection image to optionally include additional measurements on the imaged breast such as compressed thickness in order to get quantitative information about iodine or other contrast medium concentration in each point of the volume.

The double exposure occurs for each projection making up the image series, that is for each 100a, 100b, 100c, . . . , 200a, 200b, 200c . . . , 300a, 300b, 300c . . . image belonging to an image series, and therefore the result of this subtraction. The results of the double radiation exposure are illustrated in the obtained 3D model. At the end, a 3D model sequence is obtained, in which each 3D model constitutes the result of a double radiation exposure. The two acquisitions presenting different energy can be carried out by observing a certain interval after the injection of an iodized or other contrast enhancement products as shown in FIG. 6.

The variant embodiments and treatments steps described above can be applied to the double energy variant embodiment.

An embodiment of the present invention therefore provides using 3D-FFDM for contrast enhanced imaging particularly of the breast; the use of a linear reconstruction procedure allowing the quantification of the contrast uptake in each point of the volume; and the use of a non-linear reconstruction procedure allowing the optimization of the 3D-image visualization.

An embodiment of the invention provides a mammography reconstruction or tomosynthesis of the breast vascularization (the angio-mammography), which enhances an improved noise-induced contrast ratio, and also presents the advantages of a three-dimensional view. In this way an embodiment of the invention allows a localization in volume of a lesion and a finer characterization of the encountered lesion type. The height and extension of the tumors imaged can equally be identified.

Moreover, an embodiment of the invention enables illustration of the kinetic parameters of the contrast images in three dimensions, as well as enabling identification of certain kinetics typical of certain lesions. Thus, an embodiment of the invention makes it possible to evaluate the presence of multi-centric or multi-focal cancers or carcinomas. An embodiment of the invention also allows for a particularly reliable follow-up during radiotherapy.

An embodiment of the invention enables prevention of negative biopsies and avoidance of examinations via magnetic radiation (MRI), which can be a more costly procedure.

Furthermore, the method and apparatus according to an embodiment of the invention can rely on FFDM type (Full Field Digital Mammography) means for mammography, via X-rays (source and image captor) that will be used otherwise, for detection purposes and for diagnostic views.

An apparatus device according to an embodiment of the invention presents a greater accessibility than MRI imaging apparatus, due to its use of X-rays.

Moreover, an embodiment of the invention allows obtaining a visual representation of the vascularization of the breast with a spatial resolution better and/or bigger than that obtained directly by magnetic resonance imaging and with an equally satisfactory temporal resolution.

One skilled in the art may make or propose modifications to the structure and/or way and/or function and/or result and/or steps of the disclosed embodiments and equivalents thereof without departing from the scope and extant of the invention.

What is claimed is:

1. An apparatus for imaging comprising:
means for acquiring a series of images for different orientations of a means for providing radiation resulting from the attenuations of radiation across an object;
means for providing contrast-medium product to the object; and
means implementing an image treatment comprising a reconstruction of a three-dimensional model of the imaged object.

2. The apparatus in accordance with claim 1 comprising:
means for achieving two object image exposures with two beams orientated in a similar fashion and presenting different energies, leading the contrast-medium to cause different attenuations to these two energies; and
means for exploiting the attenuation difference between these two image exposures.

3. The apparatus in accordance with claim 1 comprising:
means for image retiming suitable to compensate any potential imperfections or artefacts due to undesired movement during the image acquisition.

4. The apparatus in accordance with claim 2 comprising:
means for image retiming suitable to compensate any potential imperfections or artefacts due to undesired movement during the image acquisition.

5. The apparatus in accordance with claim 3 wherein the means for retiming is implemented on the images, before the reconstruction of the three-dimensional model.

6. The apparatus in accordance with claim 4 wherein the means for retiming is implemented on the images, before the reconstruction of the three-dimensional model.

7. The apparatus in accordance with claim 3 wherein the means for retiming is implemented on the three-dimensional model after the model is reconstructed into three dimensions.

8. The apparatus in accordance with claim 6 wherein the means for retiming is implemented on the three-dimensional model after the model is reconstructed into three dimensions.

9. The apparatus according to claim 5 wherein the means for retiming is applied with respect to a mask model of the object before providing the contrast-medium to the object.

10. The apparatus according to claim 7 wherein the means for retiming is applied with respect to a mask model of the object before providing the contrast-medium to the object.

11. The apparatus in accordance with claim 1 comprising means for logarithmic treatment of the sampled attenuations to provide linear treatment of the values thus obtained, which result is represented in the three-dimensional model as reconstructed values indicative of concentrations of the contrast-medium product in different object localizations.

12. The apparatus in accordance with claim 1 comprising means for providing non-linear treatment of the acquired images to optimize contrast-enhancement visualization.

13. The apparatus in accordance with claim 1 comprising means for achieving a series of three-dimensional models, each corresponding to a different instant of the contrast enhanced image.

14. The apparatus in accordance with claim 1 comprising means for extracting contrast enhanced information by subtraction of a mask volume.

15. The apparatus in accordance with claim 1 comprising means for extracting contrast enhanced information by estimating the attenuation factor of the contrast medium using a spectral model of the image acquisition process so that the resulting image is scaled in physical units.

16. The apparatus in accordance with claim 1 comprising means for extracting contrast enhanced information by spatial filtering of 3D volumes to increase contrast to noise ratio for observations of the object.

17. The apparatus in accordance with claim 1 comprising means for extracting contrast enhanced information by computation of parametric images to characterize contrast-variation.

18. The apparatus in accordance with claim 13 comprising means for determining and displaying values of a maximum contrast growth in time, in different object localizations on the three-dimensional model.

19. The apparatus in accordance with claim 13 comprising means for determining and displaying temporal integration values of a contrast enhancement image, in different object localizations on the three-dimensional model.

20. The apparatus in accordance with claim 1 comprising means for treating the attenuations distributed in the three-dimensional model for establishing a classification of zones of the object into categories.

21. The apparatus in accordance with claim 20 wherein the means for classification identify a similarity between sampled data and data associated with a reference classification.

22. A method for imaging comprising:
acquiring a series of images for different orientations of a means for providing radiation resulting from the attenuations of radiation across an object;
providing contrast-medium product to the object; and
reconstructing a three-dimensional model of the imaged object according to claim 1.

23. A computer program comprising program code means for implementing the steps of the method according to claim 22, when the program runs on a computer.

24. A computer program product comprising a computer useable medium having computer readable program code means embodied in the medium, the computer readable program code means implementing the steps of the method according to claim 22.

25. An article of manufacture for use with a computer system, the article of manufacture comprising a computer readable medium having computer readable program code means embodied in the medium, the program code means implementing the steps of the method according to claim 22.

26. A program storage device readable by a machine tangibly embodying a program of instructions executable by the machine to perform the steps of the method according to claim 22.

27. A generated or stored signal or transmitted or a received signal, the signal embodying a program of instructions executable by a machine to perform the steps of the method according to claim 22
